(19) United States
(12) Patent Application Publication
(10) Pub. No.: US 2022/0305300 A1
(43) Pub. Date: Sep. 29, 2022

(54) BREATHING REGULATOR WITH DYNAMIC DILUTION CONTROL

(71) Applicant: Cobham Mission Systems Orchard Park Inc., Orchard Park, NY (US)

(72) Inventors: Lucas P. Mesmer, Elma, NY (US); William D. Siska, Marilla, NY (US)

(73) Assignee: Cobham Mission Systems Orchard Park Inc., Orchard Park, NY (US)

(21) Appl. No.: 17/656,108

(22) Filed: Mar. 23, 2022

Related U.S. Application Data
(60) Provisional application No. 63/167,339, filed on Mar. 29, 2021.

(51) Int. Cl. A62B 9/02; A62B 7/14

(52) U.S. Cl. CPC A62B 9/022; A62B 7/14

(57) ABSTRACT

A breathing regulator including a first stage regulator, a second stage regulator, a dilution valve, a mixing chamber, and a controller is provided. The first stage regulator is in fluid communication with pressurized source gas. The second stage regulator is in fluid communication with the first stage regulator. The dilution valve is in fluid communication with an ambient gas and includes a size-variable restriction. The mixing chamber is in fluid communication with the second stage regulator, the dilution valve, and a breathing cavity. The controller is in electrical communication with the dilution valve, the second stage regulator, and a plurality of sensors. The controller is configured to: determine a mass flow of the source gas; determine mass flow of the ambient gas; and vary the size-variable restriction of the dilution valve based on the mass flow of the source and/or the mass flow of the ambient gas.

### CROSS-REFERENCE TO RELATED APPLICATIONS

This application claims priority to U.S. Provisional Application No. 63/167,339, filed Mar. 29, 2021, and entitled “Breathing Regulator with Dynamic Dilution Control,” the entirety of which is incorporated herein by reference.

### FIELD

The present invention relates to breathing regulator devices having a variable size dilution orifice, and systems and methods for dynamic dilution control.

### BACKGROUND

In traditional dilution-demand breathing systems, the systems utilize an aneroid or similar device to control the dilution ratio between the supplied oxygen and ambient air in order to provide a mixture of ambient air and oxygen to a user. A regulator will supply oxygen as required to maintain a minimum pre-set sensing cavity pressure. In this method, assuming ambient pressure is constant, the restriction created by the limiting device is also constant. When the user inhales, the magnitude of flow varies throughout the duration of the breath while the regulator maintains a constant breathing cavity pressure. Therefore, the magnitude of airflow through the limiting device is fixed, and the remainder of the demanded gas is supplied by the oxygen source. Such a conventional device supplies variable mixtures of ambient air and oxygen to the user throughout the duration of a single breath due to the fixed size of its orifice.

### SUMMARY

The present disclosure describes systems and methods for delivering breathing gas to a person, specifically, a breathing regulator with dynamic dilution control (BRDDC). The applicant has recognized and appreciated that the size or amount of restriction between a breathing cavity and ambient air can be varied based on measurements of a mass of flow of oxygen gas and ambient air to provide a stable mixture of air and oxygen to a user. Advantageously, the dynamic dilution control described herein at least provides: (1) tighter control of targeted dilution concentration throughout all ambient pressures; (2) stable dilution concentration throughout individual or singular breaths; (3) optimized air-to-oxygen gas delivery to improve system efficiency by reducing the required oxygen supplied to the user; (4) oxygen at the start of a breath and allows the dilution restriction to open to provide ambient air for the remainder of the breath to reduce the required oxygen supplied to the user; and (5) a means of switching between dilution-demand, demand, and positive pressure regulation using feedback from various measurement devices and/or user input. Although embodiments described herein pertain to an aircraft breathing device, it should be appreciated that the systems and methods described herein can be utilized in any application requiring delivery of breathing gas to a human.

Generally, in one aspect, a breathing regulator is provided. The breathing regulator includes a first stage regulator. The first stage regulator is in fluid communication with a pressurized source gas.

The breathing regulator further includes a second stage regulator. The second stage regulator is in fluid communication with the first stage regulator.

The breathing regulator further includes a dilution valve. The dilution valve is in fluid communication with ambient gas. The dilution valve includes a size-variable restriction.

The breathing regulator further includes a mixing chamber. The mixing chamber is in fluid communication with the second stage regulator and the dilution valve.

The breathing regulator further includes a controller. The controller is in electrical communication with the dilution valve, the second stage regulator, and a plurality of sensors. The controller is configured to determine a mass flow of the pressurized source gas. The controller is further configured to determine a mass flow of the ambient gas. The controller is further configured to vary the size-variable restriction of the dilution valve. The size-variable restriction is varied based on the mass flow of the pressurized source gas and/or the mass flow of the ambient gas to provide a stable mixture of the ambient gas and the pressurized source gas to a user throughout a duration of an entire breath.

According to an example, the breathing regulator further includes an emergency bypass. The emergency bypass is in parallel fluid communication with the pressurized source gas and the first stage regulator. The emergency bypass is in fluid communication with the mixing chamber.

According to an example, the mass flow of the pressurized source gas is determined based at least in part on a setting of the second stage regulator.

According to an example, the breathing regulator further includes a first differential pressure sensor. The first differential pressure sensor is in fluid communication with the mixing chamber. The first differential pressure sensor is configured to provide a first differential pressure signal to the controller. The mass flow of the ambient gas may be determined based at least in part on the first differential pressure signal and/or a setting of the dilution valve.

According to an example, the breathing regulator further includes a second differential pressure sensor. The second differential pressure sensor is in fluid communication with a breathing cavity. The breathing cavity may be a mask. The second differential pressure sensor is configured to provide a second differential pressure signal to the controller. The breathing cavity is in fluid communication with the mixing chamber. The controller may be further configured to adjust the second stage regulator based on the first differential pressure signal, the second differential pressure signal, and/or an acceleration of the breathing regulator.

According to an example, the breathing regulator further includes an absolute pressure sensor configured to provide an absolute pressure signal to the controller, an accelerometer configured to provide an acceleration signal to the controller, and/or a temperature sensor configured to provide a temperature signal to the controller.

Generally, in another aspect, a breathing regulator control system is provided. The breathing regulator control system includes a pressurized source gas. The breathing regulator control system further includes a first stage regulator in fluid communication with the pressurized source gas. The breathing regulator control system further includes a second stage regulator in fluid communication with the first stage regulator. The breathing regulator control system further includes a dilution valve in fluid communication with ambient gas. The dilution valve includes a size-variable restriction. The breathing regulator control system further includes a mixing chamber in fluid communication with the second stage regulator and the dilution valve. The breathing regulator control system further includes a breathing cavity in fluid communication with the mixing chamber. The breathing cavity may be a mask.

The breathing regulator control system further includes a controller in electrical communication with the dilution valve, the second stage regulator, and a plurality of sensors. The controller is configured to determine a mass flow of the pressurized source gas. The controller is further configured to determine a mass flow of the ambient gas. The controller is further configured to vary the size-variable restriction of the dilution valve based on the mass flow of the pressurized source gas and/or the mass flow of the ambient gas to provide a stable mixture of the ambient gas and the pressurized source gas to a user throughout a duration of an entire breath.

Generally, in another aspect, a method for providing a stable mixture of air and oxygen to a user throughout an entire breath is provided. The method includes (1) providing, via a first stage regulator, a second stage regulator with a pressurized source gas; (2) providing, via the second stage regulator, a mixing chamber with the pressurized source gas; (3) providing, via a dilution valve, a mixing chamber with ambient gas, wherein the dilution valve comprises a size-variable restriction; (4) determining, via a controller in electrical communication with the dilution valve, the second stage regulator, and a plurality of sensors, a mass flow of the pressurized source gas; (5) determining, via the controller, a mass flow of ambient gas; (6) varying, via the controller, the size-variable restriction of the dilution valve based on the mass flow of the pressurized source gas and/or the mass flow of the ambient gas.

According to an example, the method further includes providing, via a first differential pressure sensor in fluid communication with the mixing chamber, a first differential pressure signal to the controller. The method may also include providing, via a second differential pressure sensor in fluid communication with a breathing cavity, a second differential pressure signal to the controller, wherein the breathing cavity is in fluid communication with the mixing chamber. The method may further include adjusting, via the controller, the second stage breathing regulator based on the first differential pressure signal, the second differential pressure signal, and/or an acceleration of the breathing regulator.

Generally, in a further aspect, a method for providing a stable mixture of an ambient gas and a pressurized source gas to a user throughout an entire breath is provided. The method includes (1) measuring a mass flow of the pressurized source gas; (2) measuring a mass flow of the ambient gas; (3) measuring a pressure in a breathing cavity or mask; and (4) dynamically controlling a size of a restriction between the breathing cavity or mask and the ambient gas to provide the stable mixture of the ambient gas and the pressurized source gas to the user throughout a duration of an entire breath.

According to an example, dynamically controlling the size of the restriction comprises changing an orifice opening at a start or an end of an inhalation or changing the orifice opening during a peak of the inhalation.

In an example embodiment, dynamically controlling the size of the restriction comprises decreasing an orifice opening at the start of an inhalation to decrease an amount of ambient airflow and increasing the orifice opening during a peak of the inhalation and end of the inhalation to increase the amount of ambient airflow.

In various implementations, a processor or controller can be associated with one or more storage media (generically referred to herein as “memory,” e.g., volatile and non-volatile computer memory such as ROM, RAM, PROM, EPROM, and EEPROM, floppy disks, compact disks, optical disks, magnetic tape, Flash, OTP-ROM, SSD, HDD, etc.). In some implementations, the storage media can be encoded with one or more programs that, when executed on one or more processors and/or controllers, perform at least some of the functions discussed herein. Various storage media can be fixed within a processor or controller or can be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also can appear in any disclosure incorporated by reference should be accorded a meaning most consistent with the particular concepts disclosed herein.

Other features and advantages will be apparent from the description and the claims.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various examples.

FIG. 1 is a schematic block diagram representing an example breathing regulator with dynamic dilution control system, according to aspects of the present disclosure.

FIG. 2 is a cross-sectional schematic view of an example first stage regulator of a breathing regulator with dynamic dilution control system, according to aspects of the present disclosure.

FIG. 3 is a cross-sectional schematic view of an example second stage regulator of a breathing regulator with dynamic dilution control system, according to aspects of the present disclosure.

FIG. 4 is a cross-sectional schematic view of an example emergency bypass of a breathing regulator with dynamic dilution control system, according to aspects of the present disclosure.

FIG. 5 is a cross-sectional schematic view of an example dilution valve of a breathing regulator with dynamic dilution control system, according to aspects of the present disclosure.

FIG. 6 is a cross-sectional schematic view of an example mixing chamber of a breathing regulator with dynamic dilution control system, according to aspects of the present disclosure.

FIG. 7 is a schematic representation of an example controller of a breathing regulator with dynamic dilution control system, according to aspects of the present disclosure.

FIGS. 8A and 8B are graphical plots showing the measured oxygen concentration in a breathing cavity as a function of dilution valve displacement and second stage regulator displacement, according to aspects of the present disclosure.

FIG. 9 is an example method for providing a stable mixture of air and oxygen to a user throughout an entire breath, according to aspects of the present disclosure.

FIG. 10 is a further example method for providing a stable mixture of air and oxygen to a user throughout an entire breath, according to aspects of the present disclosure.

### DETAILED DESCRIPTION

The present disclosure describes systems and methods for delivering breathing gas to a person, specifically, a breathing regulator with dynamic dilution control (BRDDC). The breathing regulator includes two electromechanically controlled valves that supply a pilot or other airborne personnel with breathing gas from an oxygen source and an ambient air source. The breathing regulator is pneumatically plumbed to a user via a breathing mask. The breathing mask has either a compensated or non-compensated inhalation and exhalation check valve. Furthermore, the breathing regulator device includes a controller interfaced with a plurality of sensors to determine the mass flows of ambient air and oxygen, the pressure in the breathing cavity or mask, the ambient pressure of the environment, the ambient temperature of the environment, and the acceleration of the breathing regulator. The breathing regulator measures the mass flow of the oxygen gas and ambient air, along with the other variables previously stated, and varies the size of the restriction between the breathing cavity and ambient air to provide a stable mixture of air and oxygen to the user throughout the duration of an entire breath.

When the breathing regulator is mounted on a breathing mask directly, an inhalation valve may not be required. The breathing regulator is capable of operating as a positive pressure breathing regulator, demand breathing regulator, and a dilution-demand breathing regulator depending on the demands of the user and the environment. While operating at positive pressure or at demand, the breathing regulator uses the controller to supply oxygen to the user at a stable positive mask gauge pressure, or negative mask gauge pressure, respectively. While operating in dilution-demand the breathing regulator uses the controller to supply a mixture of oxygen and ambient air to the user.

The breathing regulator measures the mass flow of the oxygen gas and ambient air, along with the other variables previously stated, and varies the size of the restriction between the breathing cavity and ambient air to provide a stable mixture of air and oxygen to the user throughout the duration of an entire breath. In doing so, the breathing regulator provides a number of important benefits, including, but not limited to (1) finer control of targeted dilution concentration throughout all ambient pressures, (2) stable dilution concentration throughout the duration of singular breaths, (3) improving system efficiency by reducing the required oxygen supplied to the user due to optimized air-to-oxygen gas delivery, (4) providing oxygen at the start of the breath and allowing the dilution restriction to open to provide ambient air for the remainder of the breath may reduce the required oxygen supplied to the user; (5) allowing for switching between dilution-demand, demand, and positive-pressure regulation using feedback from various measurement devices and/or user input.

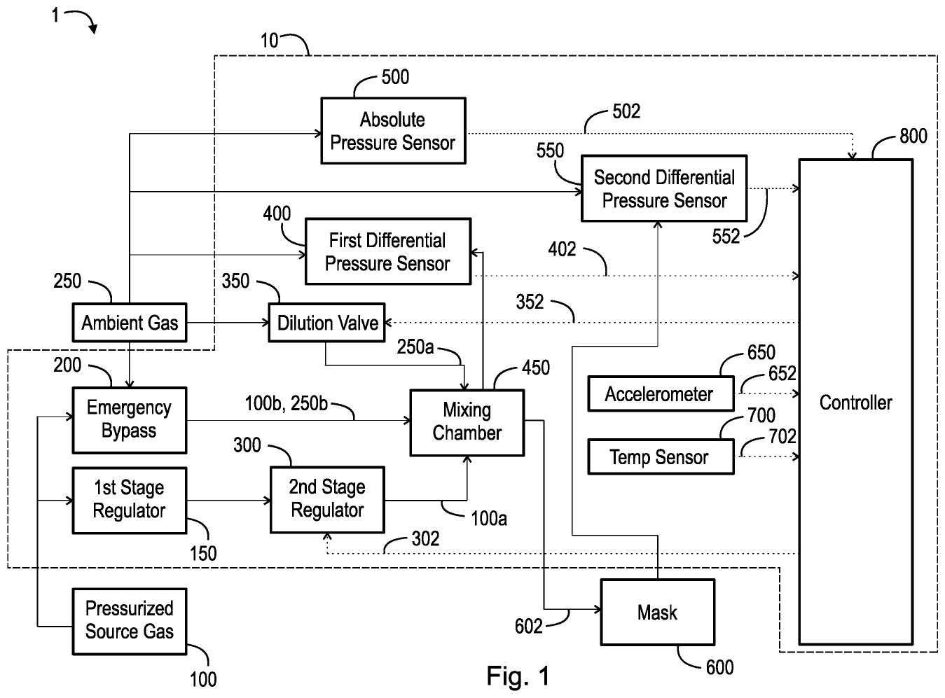

A block diagram illustrating the interactions of various aspects and components of an example breathing regulator control system 1 are shown in FIG. 1. In the example of FIG. 1, within the breathing regulator control system 1, breathing regulator 10 includes a first stage regulator 150, a second stage regulator 300, a dilution valve 350, an emergency bypass 200, a mixing chamber 450, a controller 800, and various sensors providing data to the controller 800. Regarding the sensors, the example breathing regulator 10 of FIG. 1 includes a first differential pressure sensor 400, a second differential pressure sensor 550, an absolute pressure sensor 500, an accelerometer 650, and a temperature sensor 700. In some examples, the temperature sensor 700 may be embedded into one of the differential pressure sensors 400, 550. Other types of sensors may be used in other applications.

With continued reference to FIG. 1, a pressurized source gas 100, generally oxygen or a mixture of oxygen and an inert gas such as nitrogen, is pneumatically plumbed in parallel to the first stage regulator 150 and to the emergency bypass 200. The first stage regulator 150 reduces the pressure of the pressurized source gas 100 and feeds this reduced-pressure source gas 100 to the second stage regulator 300. Similarly, ambient gas 250, generally air, is sourced to the dilution valve 350 and to the emergency bypass 200 in parallel.

The second stage regulator 300 is an electromechanical regulator which also reduces the pressure of the source gas 100, and then feeds the twice-regulated source gas 100 into the mixing chamber 450 and subsequently to the breathing cavity 600 (embodied in FIG. 1 as a mask), through which a human breathes. The dilution valve 350 is an electromechanical valve with a size-varying restriction. The dilution valve 350 allows the ambient gas 250 to enter the mixing chamber 450, combine with the source gas 100, and proceed to the mask 600.

The controller 800 is an electronic computer or microprocessor that receives information from the various sensors and then controls various aspects of the breathing regulator 10. In this example, the controller 800 receives an absolute pressure signal 502 from the absolute pressure signal 500. The absolute pressure signal 502 corresponds to the absolute pressure of the ambient gas 250 in the environment. The absolute pressure signal 502 may be used as a reference to determine the properties of the source gas 100 and the ambient gas 250, as well as to determine the altitude of the breathing regulator.

The controller 800 also receives a first differential pressure signal 402 from the first differential pressure sensor 400. The first differential pressure signal 402 corresponds to the differential pressure between the mixing chamber 450 and the ambient gas 250. The controller 800 also receives a second differential pressure signal 552 from the second differential pressure sensor 550. The second differential pressure signal 552 corresponds to the differential pressure between the mask 600 and the ambient gas 250. The controller 800 also receives an acceleration signal 652 from the accelerometer 650. The acceleration signal 652 corresponds to the acceleration of the environment (such as an airplane) and lets the controller 800 know if the environment is experiencing g-force. The controller 800 also receives a temperature signal 702 from the temperature sensor 700. The temperature signal 702 corresponds to the temperature of the environment.

The controller 800 uses these signals 402, 502, 552, 652, 702 to generate control signals 302, 352. For example, the controller 800 generates a dilution control signal 352 and a second stage regulator control signal 302. The dilution control signal 352 is provided to the dilution valve 350 to control the flow of ambient gas 250 into the mixing chamber 450. The second stage regulator control signal 302 is provided to the second stage regulator 300 to control the flow of source gas 100 into the mixing chamber 450. By controlling the dilution valve 350 and the second stage regulator 300, the controller 800 can provide the mask 600 with (1) a determined mixture of source gas 100 and ambient gas 250 at (2) a determined pressure.

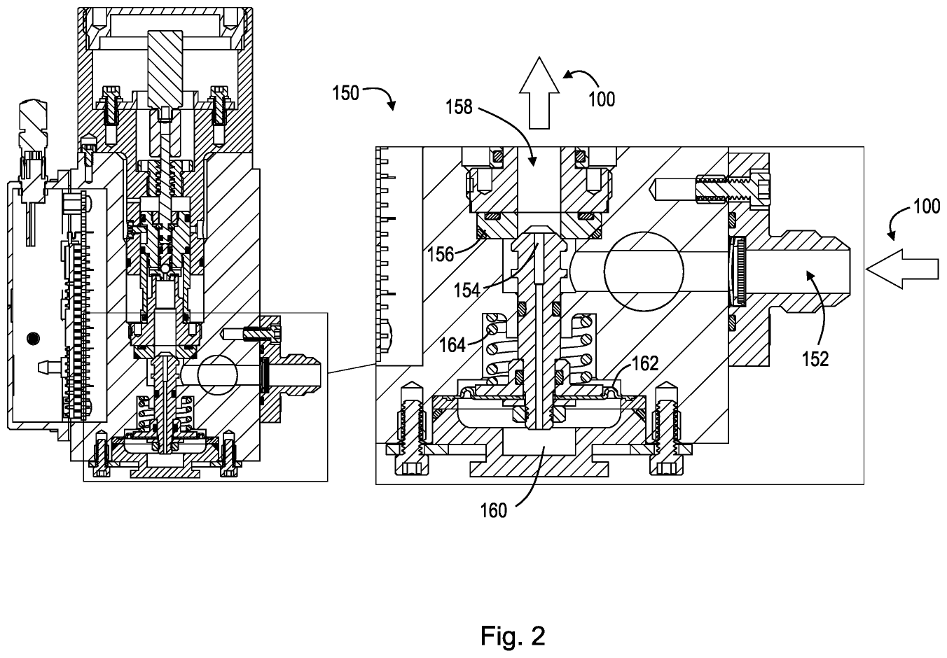

FIG. 2 shows a cross-section of the first stage regulator 150, embodied as a mechanical regulator. Pressurized source gas 100 (in one non-limiting example, 100 to 125 psi or less) enters the inlet 152 and is pneumatically plumbed to poppet 154. The poppet 154 opens and closes against seat 156. Accordingly, the pressure within outlet 158 (in one non-limiting example, approximately 20 or 25 psi) to a second stage regulator 300 (not shown) is lower than the pressure within the inlet 152. This is achieved by plumbing the outlet pressure through the poppet 154 to sensing chamber 160. The pressure in the sensing chamber 160 loads a flexible diaphragm 162. The pressure load on the flexible diaphragm 162 is balanced by the load from a helical spring 164. This balanced loading determines the pressure at the outlet 158. The poppet 154/seat 156 interface is sized to have minimal pressure loss when flowing greater than 300 standard liters per minute of oxygen. The helical spring 164 may be adjusted or calibrated to increase or decrease the pressure within outlet 158. In this example, the first stage regulator 150 is generally comprised of components traditionally associated with mechanical regulators. However, in alternative examples, the first stage regulator 150 may include non-traditional components.

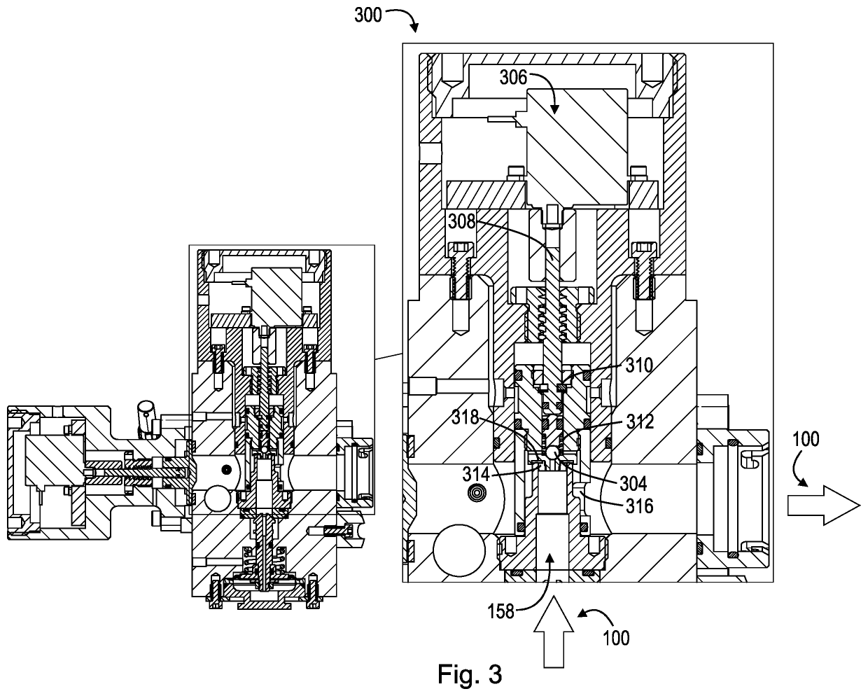

FIG. 3 shows a cross-section of the major components of the second stage regulator 300. In this example, the second stage regulator 300 is an electromechanical device that controls the delivery of pressurized source gas 100 to the mixing chamber 450 (not shown). In some examples, the pressurized source gas 100 is outputted by the second stage regulator 300 at a pressure of 1.5 psi or less. Pressurized source gas 100 is delivered to the outlet 158 of the first stage regulator 150 (not shown) where gasket 318 provides a primary seal to the mixing chamber 450. In one non-limiting example, the gasket 318 may be an elastomeric gasket. In other examples, the gasket 318 may be constructed out of any combination of a variety of materials, such as resin, thermoplastic elastomer (TPE), polytetrafluoroethylene (PTFE), etc., or plastic-like materials such as pique, etc. The embodiment of FIG. 3 features a secondary ball 304 that seals a secondary path to the mixing chamber 450. The secondary ball 304 may be constructed of a multitude of materials such as an elastomer, a resin, or a metal depending on the need of the application. A servo motor 306 drives a lead screw 308 to stroke the second stage regulator 300. A small stroke gap 310 allows the lead screw 308 to unload the secondary ball 304 while the helical spring 312 loads the gasket 318 to ensure it remains sealed throughout the initial stroke gap 310. This initial stroke gap 310, or first stage of the second stage regulator 300, enables precision control of pressurized source gas 100 delivery to the mixing chamber 450 when the human breathing on the breathing regulator control system 1 is demanding very low flows, i.e., during a low demand state. Once the stroke gap 310 is closed, the lead screw 308 lifts the gasket 318 off of the outlet 158, in the second stage of the second stage regulator 300, to achieve higher magnitudes of pressurized source gas 100 flow in medium-to-high demand states. Gas flow is further controlled by a flow limiter 314 for a portion of the total stroke, which accurately restricts the available cross-sectional area for the pressurized source gas 100 to flow. When very high source gas flows are demanded, the lead screw 308 strokes past the flow limiter 314 to achieve maximum cross-sectional area with minimal restriction. Both the first stage and second stage of the second stage regulator 300 exhaust into the mixing chamber 450 through a directional controller 316 to enable proper gas mixing.

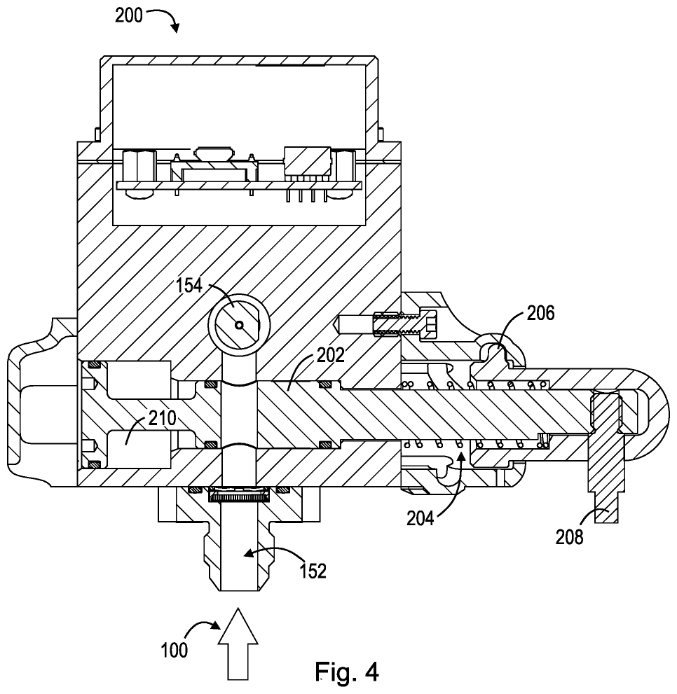

FIG. 4 shows a cross section of the major components of an emergency bypass 200. The emergency bypass 200 provides an alternative means of supplying pressurized source gas 100 as well as ambient gas 250 to the mixing chamber 450 (not shown) in the event of a failure of the first stage regulator 150 (not shown), second stage regulator 300 (not shown), and/or dilution valve 350 (not shown). For example, the mechanical nature of the emergency bypass 200 allows it to be used in case of a failure to provide electrical power to second stage regulator 300 and/or the dilution valve 350. Pressurized source gas 100 enters the inlet 152 and is pneumatically plumbed to the poppet 154 as shown in both FIGS. 2 and 4. Located between the inlet 152 and the poppet 154 is an emergency piston 202. In its normal position, the emergency piston 202 is located as shown, whereby the pressurized source gas 100 has a direct path to the poppet 154 and the ambient gas 250 is closed off to the rest of the system. Both a return spring 204 and a mechanical stop 206 prevent accidental depression of the emergency piston 202. When engaged, an operator pushes-in and turns a quarter-turn lever 208 until the mechanical stop 206 seats into a set of detents moving to the left in FIG. 4. This action disengages two of the three sealing O-rings and allows pressurized source gas 100 to flow around the emergency piston 202 and enter the emergency mixing chamber 210. Simultaneously, the emergency mixing chamber 210 is opened to ambient gas 250. Not shown in FIG. 4, the emergency mixing chamber 210 is plumbed pneumatically to the central mixing chamber 450. Accordingly, the emergency bypass 200 may be used to both provide pressurized source gas 100 and/or ambient gas 250 to the pilot, but also to allow gas within the breathing cavity 600 to escape in an over-pressure situation. Further, the two-step (push-in and quarter turn) requirement of the lever 208 prevents accidental activation of the emergency bypass 200.

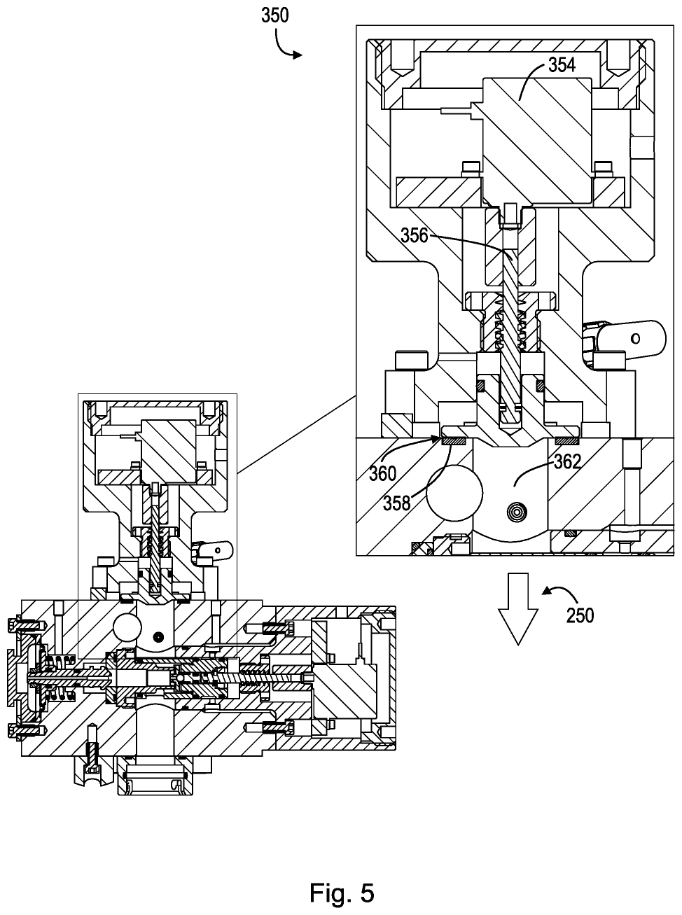

FIG. 5 shows a cross-section of the major components of a dilution valve 350. The dilution valve 350 dilutes the pressurized source gas 100 (not shown) by allowing ambient gas 250 to combine with the pressurized source gas 100 in the mixing chamber 450 (not shown). A servo motor 354 drives a lead screw 356 to stroke the dilution valve 350. As the lead screw 356 strokes, it opens a large diameter hole by unseating from a gasket 358. The gasket 358 may be an elastomeric gasket. In other examples, the gasket 358 may be constructed out of any combination of a variety of materials, such as resin, thermoplastic elastomer (TPE), polytetrafluoroethylene (PTFE), etc., or plastic-like materials such as pique, etc. Unseating the gasket 358 opens up gap 360, providing ambient gas 250 a free path to flow past the dilution valve 350 and into a dilution inlet 362 that leads to the mixing chamber 450. The dilution inlet 362 is located directionally opposite of the directional controller 314 of the second stage regulator 300 (not shown).

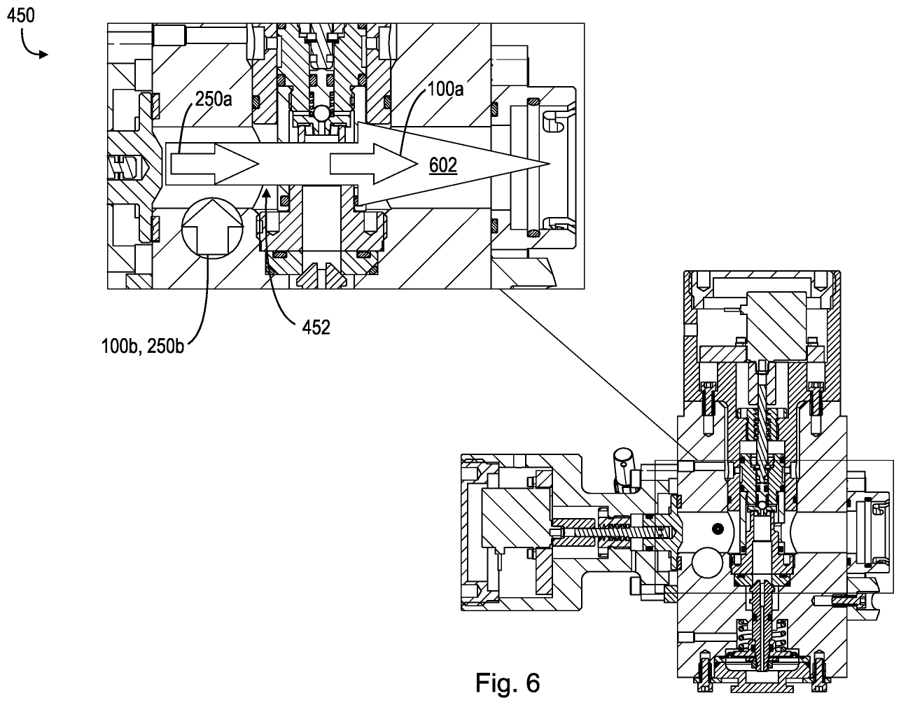

FIG. 6 shows a cross-section of the major components of a mixing chamber 450. The mixing chamber 450 is the centralized volume that allows gas from all regulators and valves to combine into a homogeneous gas before exiting the breathing regulator 10 on its way to the mask 600. The sources of each gas are shown in FIG. 6. Gas from the second stage regulator 300, and/or the emergency bypass 200, and/or the dilution valve 350 enter the central chamber 452 of the mixing chamber 450. The second stage regulator 300 adds pressurized source gas 100a, the dilution valve 350 adds ambient gas 250a, and the emergency bypass 200 (when activated) adds both pressurized source gas 100b and ambient gas 250b. These gases all combine to form an output gas 602 before proceeding to the mask 600. The directional manner in which the pressurized source gas 100a exits the second stage regulator 300 creates a venturi, or suction, effect that helps draw in ambient gas 100a from the dilution valve 350 into the mixing chamber 450.

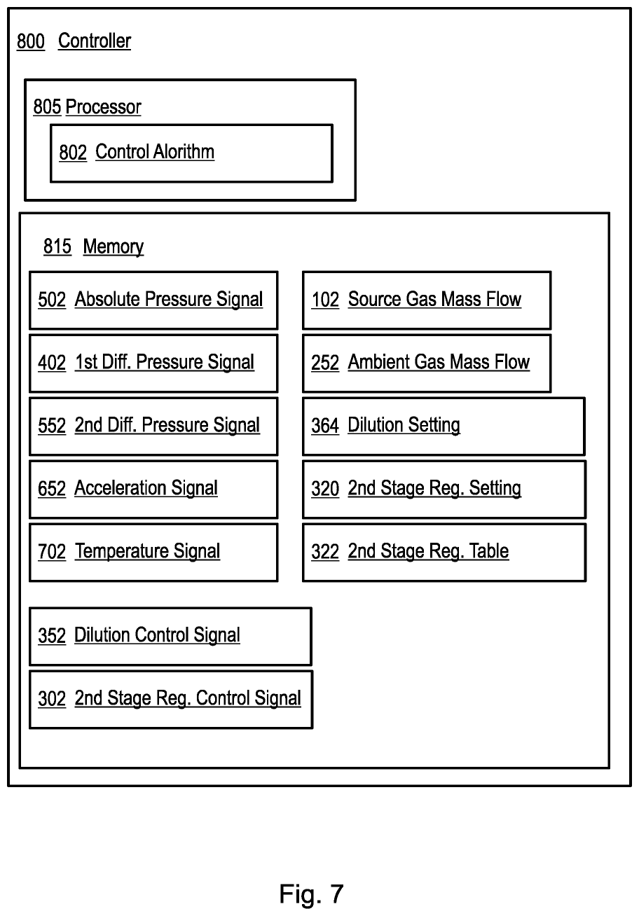

FIG. 7 shows a schematic illustration of an example controller 800. As shown in FIG. 7, the controller 800 includes a processor 805 and a memory 815. The processor 805 uses a control algorithm 802 to generate (1) a second stage regulator control signal 302 and (2) a dilution control signal 352. The second stage regulator control signal 302 is provided to the second stage regulator 300 to dynamically control the flow of source gas 100 entering mixing chamber 450. Similarly, the dilution control signal 352 is provided to the dilution valve 350 to dynamically control the flow of ambient gas 250 into the mixing chamber 450. The second stage regulator control signal 302 and the dilution control signal 352 may be stored in memory 815 following determination by the control algorithm 802.

The memory 815 may store a variety of signals received by the controller 800 from the various sensors, including a first differential pressure signal 402, a second differential pressure signal 552, an absolute pressure signal 502, an acceleration signal 652, and a temperature signal 702. The memory 815 may also store a dilution setting 364, corresponding to the size-variable restriction of the dilution valve 350, and a second stage regulator setting 320, corresponding to the stroke of the second stage regulator 300. Based on this stored data, the control algorithm may also generate a source gas mass flow 102 and an ambient gas mass flow 252. The source gas mass flow 102 and the ambient gas mass flow 252 may also be used to generate the dilution control signal 352 and the second stage regulator control signal 302.

According to an example, the absolute pressure signal 502 and the temperature signal 702 are used as part of a calculation to determine (1) the required ratio of source gas 100 to ambient gas 250 and (2) the ambient gas mass flow 252 through the dilution valve 250. The acceleration signal 652 may also be used to calculate the required ratio of source gas 100 to ambient gas 250.

The first differential pressure signal 402 corresponds to the pressure differential between the mixing chamber 450 and ambient gas 250. The first differential pressure signal 402 and the dilution setting 364 are used to calculate the ambient gas mass flow 252 into the mixing chamber 450. Similarly, the second stage regulator setting 320 is used to calculate the source gas mass flow 102 into the mixing chamber 450. In a preferred example, the source gas mass flow 102 is determined based on the position of the servo motor 306 (see FIG. 3) driving the stroke of the second stage regulator 300. A second stage regulator table 322 is stored in the memory 815 of the controller 800. The second stage regulator table 322 is populated with a plurality of servo motor positions (strokes) corresponding to mass flow values of the pressurized source gas 100. The second stage regulator setting 320 may be used to indicate the servo motor position. Thus, upon receiving the second stage regulator setting 320, the processor 805 may determine the source gas mass flow 102 by finding the mass flow value corresponding to the known servo motor position. If the servo motor position is not expressly listed in the second stage regulator table 322, the source gas mass flow 102 may be determined through interpolation of the values stored in the second stage regulator table 322. In further examples, the determined source gas mass flow 102 may be corrected for ambient pressure (according to the absolute pressure signal 502) and/or temperature (according to the temperature signal 702).

The second differential pressure signal 552 corresponds to the pressure differential between the mask 600 and the ambient gas 250 to provide pressure feedback to the control algorithm 802. In some alternative examples, the ambient gas mass flow 252 and the source gas mass flow 102 are each determined using dedicated mass flow sensors. Other pre-existing methods for determining the ambient gas mass flow 252 and/or the source gas mass flow 102 may be implemented depending on the application.

Accordingly, having received the second differential pressure signal 552, and having determined the source gas mass flow 102 and the ambient gas flow 252, the control algorithm 802 can generate a dilution control signal 352 and a second stage regulator signal 302 to adjust (1) the ratio of the source gas 100 to ambient gas 250 received by the mask 600 to meet a desired ratio and (2) the overall pressure within the mask 600 to meet a desired differential mask pressure value. In order to accurately calculate the source gas mass flow 102 and the ambient gas mass flow 252 at any dilution setting 364 or second stage regulator setting 320, the controller 800 is calibrated according to a wide range of factors. In one example, the control algorithm 802 adjusts the second stage regulator setting 320 and the dilution setting 364 at a speed greater than 100 Hz in order to achieve optimal control within the duration of a single breath. Further, the information received by the controller 800 from the various sensors allows the control algorithm 802 to configure the breathing regulator 10 as a positive-pressure breathing regulator, demand breathing regulator, a dilution-demand breathing regulator, or a dosed source gas delivery system. In even further examples, the control algorithm 802 may configure the dilution setting 364 such that dilution valve 350 operates as a relief valve when necessary.

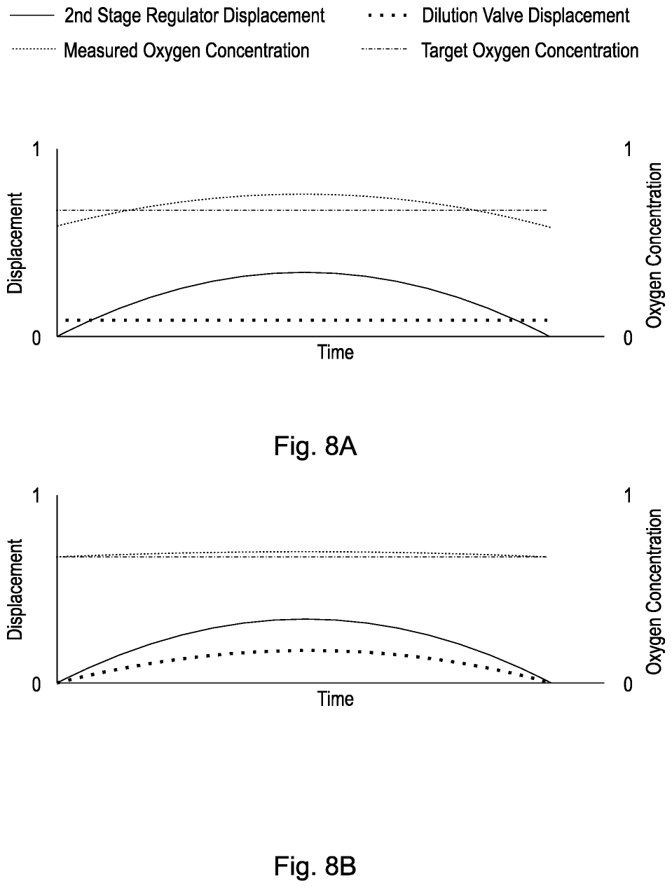

FIGS. 8A and 8B illustrate the improvement in provided oxygen concentration in conventional regulators (FIG. 8A) to the presently disclose breathing regulator (FIG. 8B) by exemplifying the effect of adjusting the dilution valve within the duration of a single breath. FIG. 8A shows the response of the system with a fixed orifice during the inhalation portion of a breath. In this example the dilution aneroid supplies too much ambient air at the start and end of inhalation and too little at the peak of inhalation, thus missing the target concentration. By contrast, FIG. 8B shows the response of the system with a dynamically controlled orifice during the inhalation portion of a breath. The dilution valve decreases the orifice opening at the start and end of inhalation to decrease ambient airflow and increases the orifice opening during the peak of inhalation to increase ambient airflow. The result is a measured oxygen concentration with less deviation from the target oxygen concentration when compared to a constant dilution orifice.

Due to the configuration of the controller and its various sensors, the breathing regulator is capable of operating as a positive pressure breathing regulator, demand breathing regulator, dilution-demand breathing regulator, or a dosed-source gas delivery system depending on the demands of the user and the environment they are exposed to. While operating at positive pressure or at demand, the breathing regulator uses the controller to supply source gas to the user at a stable positive mask differential pressure, or negative mask differential pressure, respectively. While operating in dilution-demand the breathing regulator uses the controller to supply a mixture of source gas and ambient gas to the user. In the event that the user has difficulty exhaling through the mask exhalation valve, the dilution valve is controlled to act as a relief valve to vent pressure in the tube leading to the mask. The relief functionality of the dilution valve may also aide in gas delivery by stabilizing the pressure in the mixing chamber.

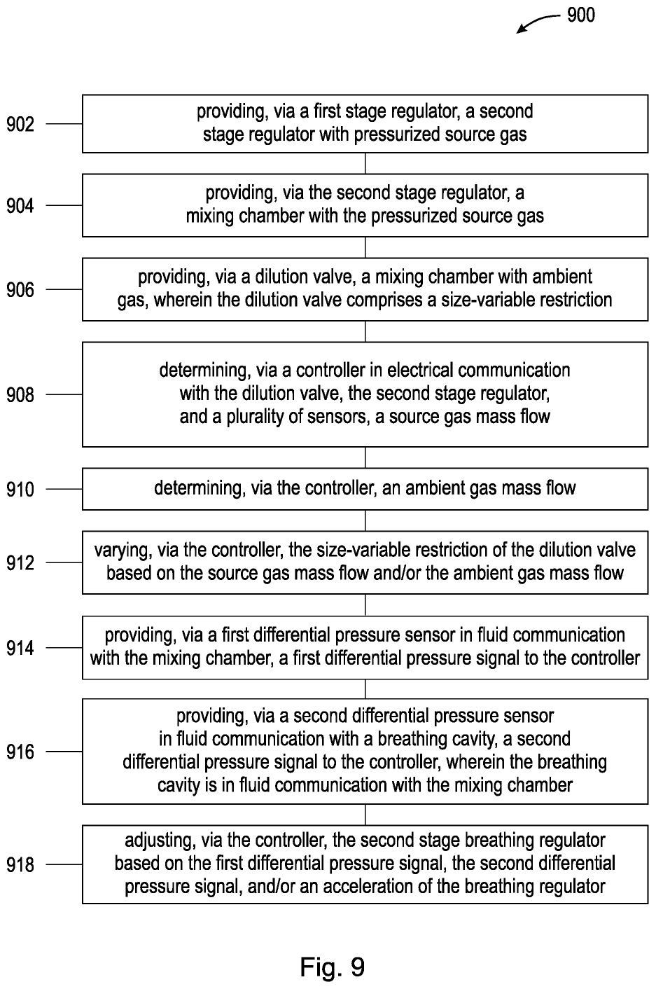

FIG. 9 illustrates an example flowchart of a method 900 for providing a stable mixture of air and oxygen to a user throughout an entire breath. The method 900 includes (1) providing 902, via a first stage regulator, a second stage regulator with pressurized source gas; (2) providing 904, via the second stage regulator, a mixing chamber with the pressurized source gas; (3) providing 906, via a dilution valve, a mixing chamber with ambient gas, wherein the dilution valve comprises a size-variable restriction; (4) determining 908, via a controller in electrical communication with the dilution valve, the second stage regulator, and a plurality of sensors, a source gas mass flow; (5) determining 910, via the controller, an ambient gas mass flow; and (6) varying 912, via the controller, the size-variable restriction of the dilution valve based on the source gas mass flow and/or the ambient gas mass flow.

According to an example, the method 900 further includes providing 914, via a first differential pressure sensor in fluid communication with the mixing chamber, a first differential pressure signal to the controller. The method 900 may also include providing 916, via a second differential pressure sensor in fluid communication with a breathing cavity, a second differential pressure signal to the controller, wherein the breathing cavity is in fluid communication with the mixing chamber. The method 900 may further include adjusting 918, via the controller, the second stage breathing regulator based on the first differential pressure signal, the second differential pressure signal, and/or an acceleration of the breathing regulator.

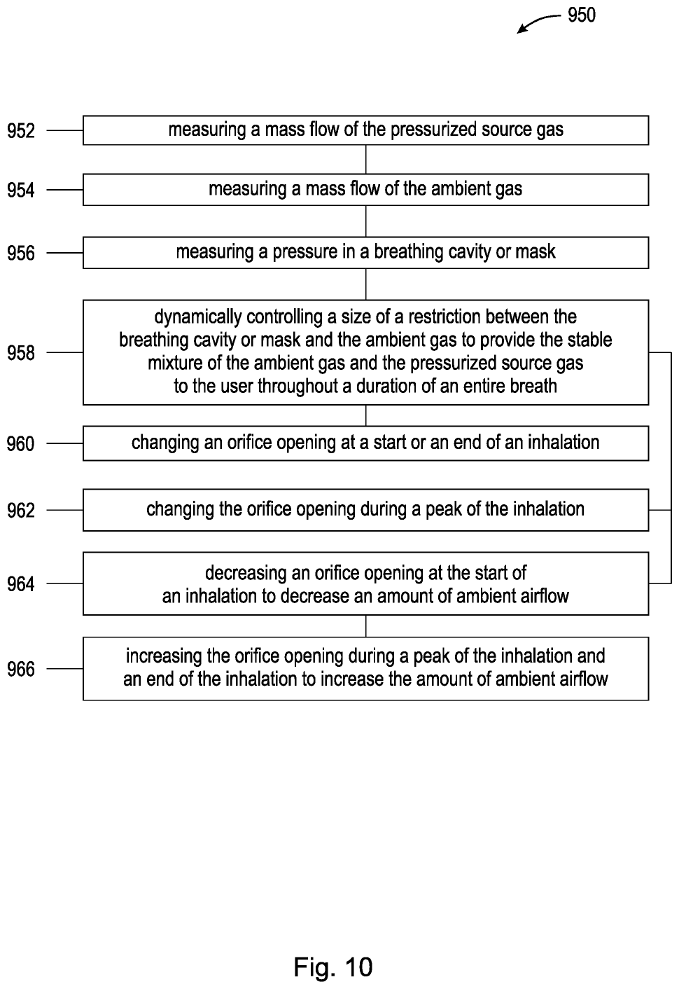

FIG. 10 illustrates a further example flowchart of a method 950 for providing a stable mixture of air and oxygen to a user throughout an entire breath. The method 950 includes (1) measuring 952 a mass flow of the pressurized source gas; (2) measuring 954 a mass flow of the ambient gas; (3) measuring 956 a pressure in a breathing cavity or mask; and (4) dynamically controlling 958 a size of a restriction between the breathing cavity or mask and the ambient gas to provide the stable mixture of the ambient gas and the pressurized source gas to the user throughout a duration of an entire breath.

According to an example, dynamically controlling 958 the size of the restriction comprises changing 960 an orifice opening at a start or an end of an inhalation or changing 962 the orifice opening during a peak of the inhalation.

In an example embodiment, dynamically controlling 958 the size of the restriction comprises decreasing 964 an orifice opening at the start of an inhalation to decrease an amount of ambient airflow and increasing 966 the orifice opening during a peak and an end of the inhalation to increase the amount of ambient airflow.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some examples, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific examples described herein. It is, therefore, to be understood that the foregoing examples are presented by way of example only and that, within the scope of the appended claims and equivalents thereto, examples can be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A breathing regulator, comprising:
a first stage regulator in fluid communication with a pressurized source gas;
a second stage regulator in fluid communication with the first stage regulator;
a dilution valve in fluid communication with ambient gas, wherein the dilution valve comprises a size-variable restriction;
a mixing chamber in fluid communication with the second stage regulator and the dilution valve;
a controller in electrical communication with the dilution valve, the second stage regulator, and a plurality of sensors, wherein the controller is configured to:
determine a mass flow of the pressurized source gas;
determine a mass flow of the ambient gas; and
vary the size-variable restriction of the dilution valve based on the mass flow of the pressurized source gas and/or the mass flow of the ambient gas to provide a stable mixture of the ambient gas and the pressurized source gas to a user throughout a duration of an entire breath.

2. The breathing regulator of claim 1, further comprising an emergency bypass in parallel fluid communication with the pressurized source gas and the first stage regulator, and in fluid communication with the mixing chamber.

3. The breathing regulator of claim 3, wherein the mass flow of the pressurized source gas is determined based at least in part on a setting of the second stage regulator.

4. The breathing regulator of claim 1, further comprising a first differential pressure sensor in fluid communication with the mixing chamber and configured to provide a first differential pressure signal to the controller.

5. The breathing regulator of claim 4, wherein the mass flow of the ambient gas is determined based at least in part on the first differential pressure signal and/or a setting of the dilution valve.

6. The breathing regulator of claim 4, further comprising a second differential pressure sensor in fluid communication with a breathing cavity and configured to provide a second differential pressure signal to the controller, wherein the breathing cavity is in fluid communication with the mixing chamber.

7. The breathing regulator of claim 6, wherein the controller is further configured to adjust the second stage regulator based on the first differential pressure signal, the second differential pressure signal, and/or an acceleration of the breathing regulator.

8. The breathing regulator of claim 6, wherein the breathing cavity is a mask.

9. The breathing regulator of claim 1, further comprising an absolute pressure sensor configured to provide an absolute pressure signal to the controller, an accelerometer configured to provide an acceleration signal to the controller, and/or a temperature sensor configured to provide a temperature signal to the controller.

10. A breathing regulator control system, comprising:
a pressurized source gas;
a first stage regulator in fluid communication with the pressurized source gas;
a second stage regulator in fluid communication with the first stage regulator;
a dilution valve in fluid communication with ambient gas, wherein the dilution valve comprises a size-variable restriction;
a mixing chamber in fluid communication with the second stage regulator and the dilution valve;
a breathing cavity in fluid communication with the mixing chamber;
a controller in electrical communication with the dilution valve, the second stage regulator, and a plurality of sensors, wherein the controller is configured to:
determine a mass flow of the pressurized source gas;
determine a mass flow of an ambient gas; and
vary the size-variable restriction of the dilution valve based on the mass flow of the pressurized source gas and/or the mass flow of the ambient gas to provide a stable mixture of the ambient gas and the pressurized source gas to a user throughout a duration of an entire breath.

11. The breathing regulator control system of claim 12, wherein the breathing cavity is a mask.

12. A method for providing a stable mixture of air and oxygen to a user throughout an entire breath, comprising:
providing, via a first stage regulator, a second stage regulator with a pressurized source gas;
providing, via the second stage regulator, a mixing chamber with the pressurized source gas;
providing, via a dilution valve, a mixing chamber with ambient gas, wherein the dilution valve comprises a size-variable restriction;
determining, via a controller in electrical communication with the dilution valve, the second stage regulator, and a plurality of sensors, a mass flow of the pressurized source gas;
determining, via the controller, a mass flow of ambient gas;
varying, via the controller, the size-variable restriction of the dilution valve based on the mass flow of the pressurized source gas and/or the mass flow of the ambient gas.

13. The method of claim 12, further comprising providing, via a first differential pressure sensor in fluid communication with the mixing chamber, a first differential pressure signal to the controller.

14. The method of claim 13, further comprising providing, via a second differential pressure sensor in fluid communication with a breathing cavity, a second differential pressure signal to the controller, wherein the breathing cavity is in fluid communication with the mixing chamber.

15. The method of claim 14, further comprising adjusting, via the controller, the second stage breathing regulator based on the first differential pressure signal, the second differential pressure signal, and/or an acceleration of the breathing regulator.

16. A method for providing a stable mixture of an ambient gas and a pressurized source gas to a user throughout an entire breath, comprising:
measuring a mass flow of the pressurized source gas;
measuring a mass flow of the ambient gas;
measuring a pressure in a breathing cavity or mask;
dynamically controlling a size of a restriction between the breathing cavity or mask and the ambient gas to provide the stable mixture of ambient gas and pressurized source gas to the user throughout a duration of an entire breath.

17. The method of claim 16, wherein dynamically controlling the size of the restriction comprises changing an orifice opening at a start or an end of an inhalation or changing the orifice opening during a peak of the inhalation.
